(19) United States
(12) Patent Application Publication
(10) Pub. No.: US 2021/0098092 A1
(43) Pub. Date: Apr. 1, 2021

(54) PRIVACY-PRESERVING MEDICAL SEARCH SYSTEM USING SIMILARITY PRESERVING HASHING

(71) Applicant: KONINKLIJKE PHILIPS N.V., Eindhoven (NL)

(72) Inventors: Gajendra Jung Katuwal, Somerville, MA (US); Bishal Lamichhane, Eindhoven (NL); Mohammad Shahed Sorower, Natick, MA (US)

(21) Appl. No.: 17/032,019

(22) Filed: Sep. 25, 2020

Related U.S. Application Data
(60) Provisional application No. 62/906,165, filed on Sep. 26, 2019.

(51) Int. Cl. G16H 10/60; G06F 21/62; G06F 16/14

(52) U.S. Cl. CPC G16H 10/60; G06F 21/6245; G16H 50/70; G06F 16/152; G06F 16/156

(57) ABSTRACT

Implementations set forth herein relate to a peer-to-peer search system for patient medical records for leveraging benefits of identifying similar patient cases in a secured singular network. The peer-to-peer search system can use a distributed ledger to securely correlate similar instances of medical data, located in various other systems, to a globally accessible network that is available via the peer-to-peer search system. For instance, medical data from various sources can be hashed by a hash technique, such as locality-sensitive hashing, and stored in a hash database. When the hash database is queried via the peer-to-peer search system, hash data corresponding to query results can be provided and, optionally, ranked according to similarities between a hashing of input query to a hashing of documents embodying the query results.

### BACKGROUND

Privacy of medical records remains a crucial feature of healthcare systems across the globe. However, medical professionals may require access to a variety of records in order to treat patients and conduct research. Oftentimes, medical professionals may only have access to medical records that have been stored and/or generated at a local facility, but those local medical records may not be comprehensive with respect to a particular patient and/or condition. Rather, a patient, and/or similar patients, may have been treated at various facilities that have different protocols for accessing medical records. As a result, an inquiring medical professional may have to expend valuable resources in order to collect the medical records—which can detract from patient care and may not result in the medical professional receiving all available records.

### SUMMARY

Implementations set forth herein relates to systems, methods, and apparatuses for providing a decentralized search system for finding similar medical records stored in disparate healthcare networks across the globe. Using this system, medical professionals can perform research by leveraging conveniences of a globally supplied database/indexing, without compromising privacy of patients whose records have contributed to the information accessible via the database. Self-preserving hashing methods are applied on medical records to create search indices, which enables the inter-network search of medical records without compromising their privacy. In order to generate information for the database various different medical records can be processed according to one or more hashing functions in order to generate hashed medical data. The hashed medical data can be generated according to, for example, a locality preserving hashing function and/or a similarity preserving hashing function. The one or more hashing functions can be accessible to participants of the decentralized search system, thereby allowing input queries to be hashed according to similar methods.

The hashed medical data or search database containing search indices (hashed values) can be stored in centralized servers or a decentralized system such as a peer-to-peer network or a hybrid of both. The search database can be globally accessible via an interface of an application executing at a computing device that has network connectivity with the database. In some implementations, a peer-to-peer network can be used to allow client devices providing queries to be connected with the database of hashed medical data. As an example, an application executing at a hospital computing device that is approved for accessing the search system can include a search interface, a medical profile interface, and/or a medical record interface. The medical record interface can provide an interface through which medical professionals and other users can create and/or modify various medical records for patients associated with an organization that manages the medical records. The medical profile interface can provide an interface through which medical professionals and/or other users can create and/or modify profiles for patients that correspond to medical records managed by the organization. The medical profiles and medical records for each organization approved for the search system can be hashed according to one or more of the same or different hashing functions, which can optionally be employed by other organizations.

In some implementations, a search interface for the search system can be accessible via a local client device, such as a personal computing device located at a location for a particular organization. A search query can be provided in any data format such as text, image, audio, and/or any other format of data. When a user provides a search query, such as a medical image, to the search interface, the search query can be hashed using one or more hashing functions, such as a similarity preserving hashing function that was previously used to hash one or more medical records and/or medical profiles generated at one or more other organizations. The resulting hashed search query can then be compared to hashed medical data that is stored in the database and/or provided by one or more other organizations. For example, a user at a first local network can be accessing an application for querying the search system. The search system can provide access to hashed medical data created at a second local network and a third local network. In response to a query from the user at the first local network, the input search query can be hashed, and the hashed query data can be compared to the hashed medical data available at the search system database. Query results can then be indicated to the user according to how similar each hashed medical record is to the hashed query data.

In some implementations, changes to medical records that were used to generate the hashed medical data can be tracked using a distributed ledger (e.g., blockchain), in order that various versions of a hashed medical record can be searched in response to a search query. For instance, hashed medical data stored in a database can include a hashing of a first medical record for a first patient and a separate hashing of a second medical record for a second patient. The first medical record can be generated at a first computing device that is connected to a first local area network, and the second medical record can be generated at a second computing device that is connected to a second local area network that is different from the first local area network. When a medical professional accesses the first medical record and modifies the first medical record, for example, based on receiving test results and/or a new diagnosis for the first patient, the hashing of the first medical record can be updated. In some implementations, the hashing of the first medical record can characterize a transaction in which the medical professional modified the first medical record. In this way, the hashing of the first medical record can include information characterizing a previous version of the first medical record and a current version of the first medical record, and optionally transactional data characterizing a context in which the medical professional made changes to the first medical record.

In some implementations, when a user provides a query that causes none or some query results to be rendered, but the rendered query results do not exhibit a threshold similarity to the query, the query can be stored as hashed query data at the database. As a result, when another query that is similar to the previous query is submitted to the search system from a different medical professional, the subsequent query results can identify the previous query. This can allow medical professionals to be put into contact with each other based on common research interests. Furthermore, should additional hashed medical data that matches the previous query be submitted to the database, each medical professional that submitted the previous query and/or the other query can be notified of the additional hashed medical data. In this way, medical professionals can be promptly notified of medical data that may be helpful to their research, thereby expediting diagnosis and treatment of patients that are served by the research.

The term “network” as used herein refers to any interconnection of two or more devices (including controllers or processors) that facilitates the transport of information (e.g., for device control, data storage, data exchange, etc.) between any two or more devices and/or among multiple devices coupled to the network. As should be readily appreciated, various implementations of networks suitable for interconnecting multiple devices may include any of a variety of network topologies and employ any of a variety of communication protocols. Additionally, in various networks according to the present disclosure, any one connection between two devices may represent a dedicated connection between the two systems, or alternatively a non-dedicated connection. In addition to carrying information intended for the two devices, such a non-dedicated connection may carry information not necessarily intended for either of the two devices (e.g., an open network connection). Furthermore, it should be readily appreciated that various networks of devices as discussed herein may employ one or more wireless, wire/cable, and/or fiber optic links to facilitate information transport throughout the network.

The term “user interface” as used herein refers to an interface between a human user or operator and one or more devices that enables communication between the user and the device(s). Examples of user interfaces that may be employed in various implementations of the present disclosure include, but are not limited to, switches, potentiometers, buttons, dials, sliders, a mouse, keyboard, keypad, various types of game controllers (e.g., joysticks), track balls, display screens, various types of graphical user interfaces (GUIs), touch screens, microphones and other types of sensors that may receive some form of human-generated stimulus and generate a signal in response thereto.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 illustrates a view of a system for providing hashed medical data to a database of a search system that preserves the privacy of patients while allowing approved entities to use the search system to identify similarities between medical records.

FIG. 2A and FIG. 2B illustrate views of a user accessing a search system database that preserves privacy of correlated patients while providing search functionality to approved users.

FIG. 3 illustrates a system for providing a search system that allows users to search hashed medical data while preserving privacy of patients whose data was hashed in order to create the hashed medical data.

FIG. 4 illustrates a method for creating a search system that allows users to search hashed medical record data while preserving privacy of patients whose data has contributed to the search system.

FIG. 5 is a block diagram of an example computer system.

### DETAILED DESCRIPTION

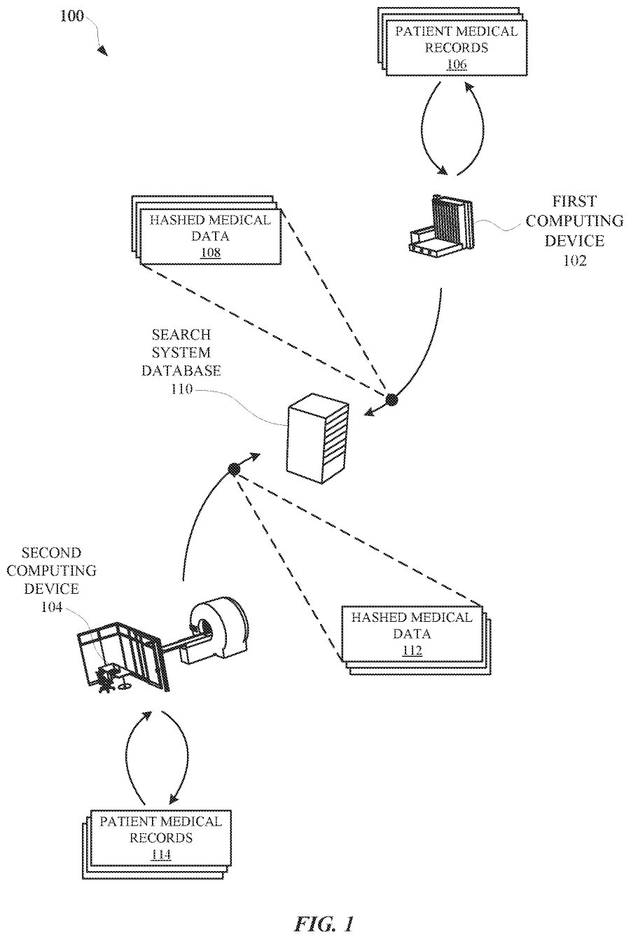

FIG. 1 illustrates a view 100 of a system for providing hashed medical data to a database of a search system that preserves the privacy of patients while allowing approved entities to use the search system to identify similarities between medical records. The system can include a search system database 110, which can comprise hashed medical data that characterizes encrypted medical records and/or medical profiles from a variety of different sources. The medical records, which can include medical profiles, can be provided by different computing devices that are connected to different local area networks when the medical records were generated. As an example, patient medical records 106 can be generated at the first computing device 102, and patient medical records 114 can be generated at a second computing device 104. In some implementations, the search system database 110 can be a distributed database that is decentralized relative to client devices that have been provided access to the database 110. However, in other implementations, the search system database 110 can be stored at one or more centralized databases.

In some implementations, each computing device of the first computing device 102 and the second computing device 104 can hash medical records to generated hashed medical data, and thereafter transmit the hashed medical data to the search system database 110. Additionally, or alternatively, each computing device can provide access to hashed medical data without providing a copy of some or all of the hashed medical data to the search system database 110. The hashed medical data can be generated from any source of medical data, such as stored digital files, sensor signals, and/or any other medium through which information can be communicated.

Each computing device can be registered with the search system database 110 and/or can include an application for accessing search features for performing medical research via the search system database 110. As an example, the first computing device 102 can be located in a hospital and can provide access to the application. When a user accesses the application, the user can navigate to a search interface for providing search queries to the search system database 110. In some response to receiving a search query, the search system database 110 can identify hashed medical data that is most related to the search query. In response, the application can identify search query results to the user, and, thereafter, the user can access certain related hashed medical data, at least with prior permission from owners of the related hashed medical data. In some implementations, medical data can be processed to generate an embedding space that is based on medical data from various different patients from various geographic regions. For instance, the medical data can be projected into a latent space, wherein clusters of data can have a Euclidian distance that is based on a degree of similarity between information characterized by the data.

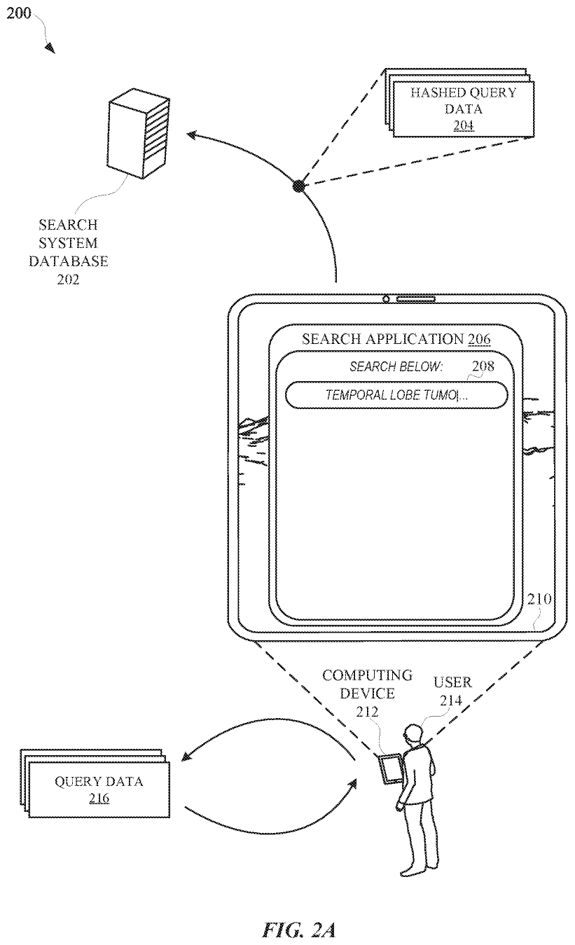

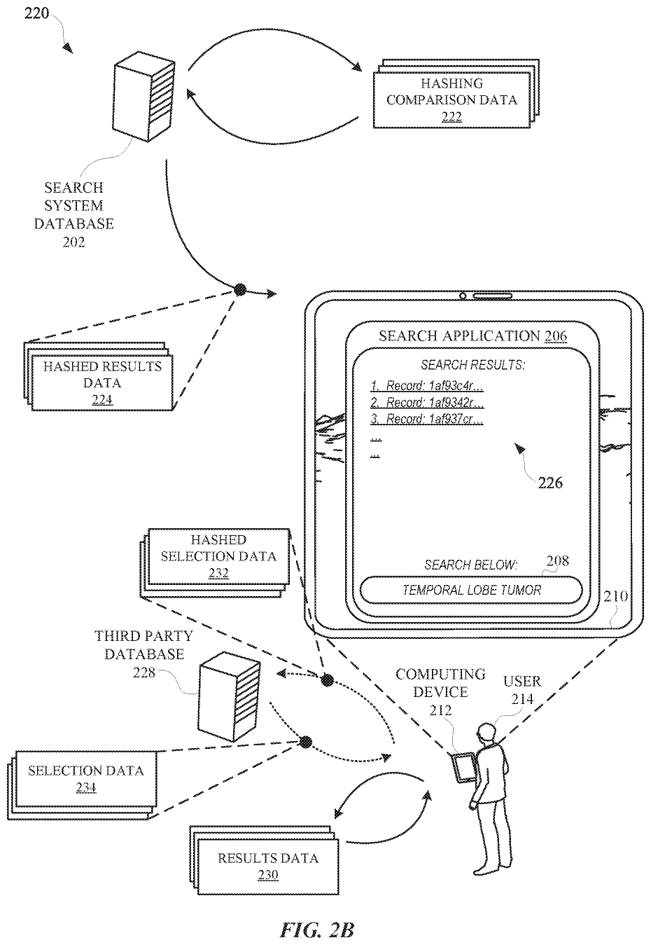

FIG. 2A and FIG. 2B illustrate a view 200 and a view 220 of a user 214 accessing a search system database 202 that preserves privacy of correlated patients while providing search functionality to approved users. The user 214 can provide a search input 208 to a computing device 212 in order to initialize execution of a search query at the search system database 202. The search input 208 can be, for example, “temporal lobe tumor,” which can refer to a condition of a patient who the user 214 is assisting. The search input 208 can be provided to a search application 206 that is associated with the search system database 202 and provides a secure medium through which the user 214 can communicate with the search system database 202.

As an example, in response to the search application 206 receiving the search input 208, the search application 206 can generate query data 216, which can be processed at the computing device 212. Processing of the query data 216 can include converting the query data 216 into hashed query data 204 according to one or more hashing functions. The one or more hashing functions can be the same as one or more hashing functions used to generate hashed medical data that is accessible via the search system database 202. For example, the one or more hashing functions can include one or more similarity preserving hashing functions that that preserves similarities of similarly arranged characters in a particular portion of data that exhibits the similarities. For instance, hashing a string of characters such as “temporal lobe tumor” according to a similarity preserving hashing function can result in a hashing value of “1pYbP8t3n0tfSyCDd.” Furthermore, this resulting hashing value can be occur in hashed medical data that includes the phrase “temporal lobe tumor” regardless of any other characters surrounding the phrase (e.g., “the patient has a temporal lobe tumor . . . ” or “MRI reveals temporal lobe tumor on right side . . . ”).

The hashed query data 204 can be transmitted to the search system database 202 over a secure network in order that the search system database 202 can provide search query results, as illustrated in view 220 of FIG. 2B. In response to the user 214 submitting the search input 208 to the search system database 202 via the search application 206, the computing device 212 can generate the hashed query data 204 and transmit the hashed query data 204 to the search system database 202. The hashed query data 204 can be transmitted to the search system database 202 via a wide area network such as the internet.

When the search system database 202 received the hashed query data 204, the search system database 202 and/or one or processors that interact with the search system database 202, can generate hashing comparison data 222. The hashing comparison data 222 can characterize one or more similarities between the hashed query data 204 and hashed medical data that is accessible via the search system database 202. In some implementations, because each instance of data is hashed according to one or more of the same similarity preserving hashing functions, each instance of hashed data can exhibit similar characteristics when instances of non-hashed data exhibit similar characteristics. Furthermore, these similar characteristics can be characterized by the hashing comparison data 222.

In some implementations, patients that have undergone similar testing procedures, such as an MRI scan of their temporal lobe, will have corresponding medical records characterizing those testing procedures. As a result, when those medical records are hashed according to one or more similarity preserving hashing functions, the resulting hashed medical data will have common characters and/or common arrangements of characters. As an example, based on the search input 208 being executed, the search system database 202 can be used to generate the hashing comparison data 222. Thereafter, the one or more processors in communication with the search system database 202 can generate hashed results data 224, which can include identifiers for hashed medical data that is most relevant to the natural language content of the search input 208.

When the search application 206 receives the hashed results data 224, the search application 206 can identify an owner of the hashed results data 224 in order to generate results data 230. For instance, a distributed ledger can be available for allowing the user to identify and validate an owner of medical data corresponding to the hashed results data 224. The search application 206 can then cause a graphical user interface 210 of the computing device 212 to render search results 226 using the results data 230. In some implementations, the search results 226 can include hashing values corresponding to medical records that are most relevant to the search input 208, “temporal lobe tumor.” The user 214 can then select a particular result (e.g., “1. Record: 1af93c4r . . . ”) in order to determine whether to user the particular result for further research and/or assisting a patient. In some implementations, as part of being approved to user 214 to use the search system database 202, the user 214 can select a particular result of the search results 226 and review content of a medical profile and/or a medical record that the particular result is based on or otherwise corresponds to. For example, the particular result can correspond to a different patient in another country and can be selected by the user 214 in order to be granted an ability to review a particular medical record for the patient (e.g., a medical record describing how the particular patient was treated for a temporal lobe tumor).

Additionally, or alternatively, the computing device 212 can receive the hashed results data 224 and, in response to the user 214 selecting a particular result, the search application 206 can transmit a request characterizing the selection of the particular result. Specifically, the search application 206 can identify a source of a medical record corresponding to the particular result using data provided by the search system database 202. Based on this data, the search application 206 can identify a third party database 228 for requesting supplemental selection data 234. In other words, the search system database 202 can generate hashed selection data 232 and transmit the hashed selection data 232 to the identified third party database 228. When one or more processors that manage the third party database 228 receives the hashed selection data 232, the third party database 228 can identify selection data 234 and provide the selection data 234 to the computing device 212.

In some implementations, the selection data 234 can include hashed medical data that corresponds to the selection of a search result. When the selection data 234 include hashed medical data, the search application 206 and/or the computing device 212 can generate corresponding medical data using one or more hashing functions. Additionally, or alternatively, when the selection data 234 includes medical data, the search application 206 and/or the computing device 212 can render the medical data at the graphical user interface 210. In this way, the user 214 is able to perform research without comprising patient privacy for patients whose records would not otherwise be accessible to the user 214.

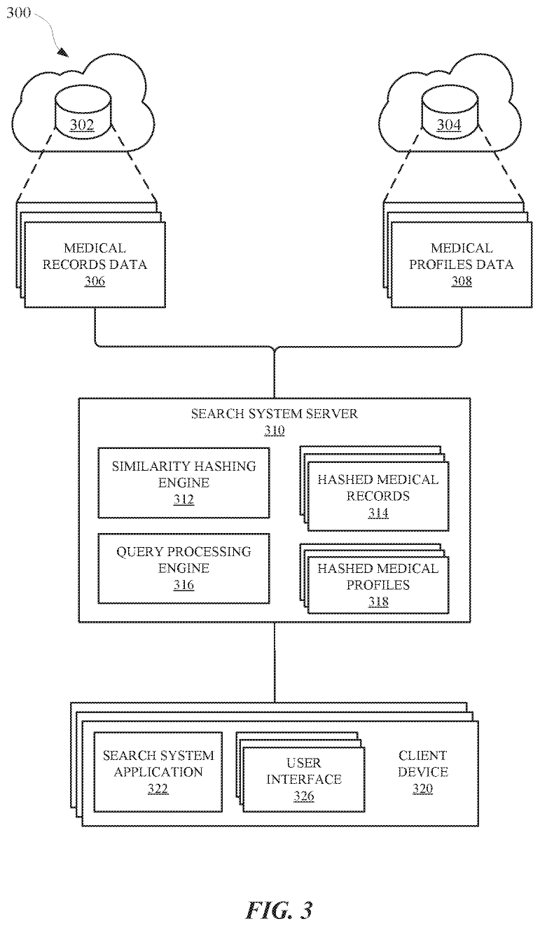

FIG. 3 illustrates a system 300 for providing a search system that allows users to search hashed medical data while preserving privacy of patients whose data was hashed in order to create the hashed medical data. The system 300 can include multiple different databases, such as a first database 302 for storing medical records data 306 and a second database 304 for storing medical profiles data 308. A search system server 310 can be in communication with one or more databases that include the first database 302 and the second database 304. In some implementations, the search system server 310 can include a similarity hashing engine 312, which can operate to hash the medical records data 306 and/or the medical profiles data 308. However, in some implementations, each database can hash their own medical data prior to the search system server 310 being provided access to the medical data.

In some implementations, the system 300 can include one or more client devices 320 that can provide access to a search system application 322 for performing searches of hashed medical records 314 and/or hashed medical profiles 318. For instance, a client device 320 can include a search system application 322 with a user interface 326 that includes a search field, in which a user can submit search queries. When a user submits a search query to the user interface 326 (e.g., by providing a search character string, a spoken input, and/or a file), the search query can be hashed at the client device 320 and/or hashed at a query processing engine 316 of the search system server 310. Using one or more processors at the search system server 310 and/or one or more other computing devices, hashed query data can be compared to hashed medical records 314 and/or hashed medical profiles 318. When the one or more processors determine that there is at least a threshold similarity and/or correspondence between the hashed query data and the hashed medical records 314 and/or hashed medical profiles 318, the search system server 310 can generated hashed results data. The hashed results data can then be shared with the client device 320 that submitted the search query.

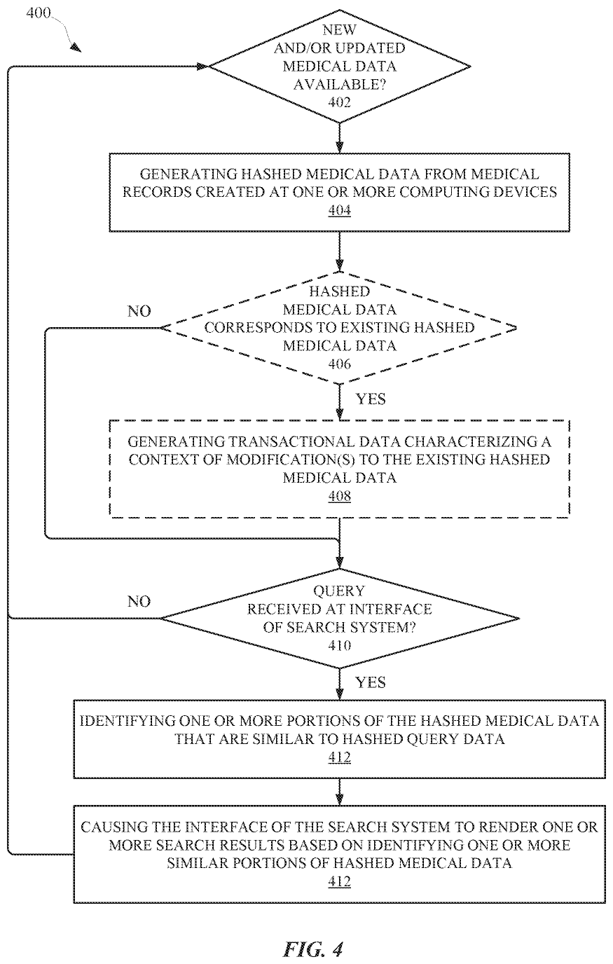

FIG. 4 illustrates a method 400 for creating a search system that allows users to search hashed medical record data while preserving privacy of patients whose data has contributed to the search system. The method 400 can be performed by one or more computing devices, applications, and/or any other apparatus for module capable of accessing medical data. The method 400 can include an operation 402 of determining whether any new and/or updated medical data is available to the search system. The search system can include a database that is accessible to various participants and/or other approved entities, and each entity can provide hashed medical data to the database. For example, at least one entity can be a hospital that includes a network of computing devices for generating medical data for patients. In order to supplement information in the database, one or more computing devices of the network of computing devices can hash the generated medical data and supply the hashed medical data to the database of the search system. In some implementations, hashing can be performed using a similarity preserving hashing function. Furthermore, the resulting hashed medical data can be a predetermined size (e.g., character length) for each medical record that is hashed using the similarity preserving hashing function.

The method 400 can proceed from the operation 402 to an optional operation 404 when new and/or updated medical data is available. New and/or updated medical data can refer to hashed medical data that is provided by one or more third-party entities relative to the entity that provides a search system interface for searching the database. The operation 404 can include generating hashed medical data from medical records created at one or more computing devices. For example, in some implementations, a third party entity can provide access to a medical record prior to hashing of the medical record. Thereafter, the search system can use the similarity preserving hashing function to generate hashed medical data from the medical record. Alternatively, or additionally, a third-party entity can use a hashing function to generate hashed medical data and provide the hashed medical data to the database without the database having access to the non-hashed medical data.

The method 400 can proceed from the operation 402 and/or the optional operation 404 to an optional operation 406. The operation 406 can include determining whether hashed medical data corresponds to any available hashed medical data. In other words, the search system can determine whether recently provided hashed medical data (e.g., for a new medical record) corresponds to a previously provided portion of hashed medical data (e.g., for an existing medical record). Existing hashed medical data can “correspond” to received-hashed medical data when the medical data refers to the same or similar patient, the same or similar diagnosis, the same or similar doctor, the same or similar treatment, and/or any other feature of a medical profile and/or a medical record. When the received hashed medical data is determined to not correspond to an existing portion of hashed medical data, the method 400 can proceed from the optional operation 406 to an operation 410. Alternatively, when the received hashed medical data is determined to correspond to an existing portion of hashed medical data, the method 400 can proceed to an optional operation 408.

The operation 408 can include generating transactional data characterizing a context of one or more modifications to the existing hashed medical data. For example, when the received hashed medical data is determined to be correlated to existing hashed medical data, a context in which the received hashed medical data was modified and/or updated can be determined. For example, existing hashed medical data can correspond to a first version of a medical record for a patient and the received hashed medical data can correspond to a second version of the medical record for the patient. A difference between the first version and the second version can be determined based on information accessible via a third party entity that provided the existing hashed medical data and the received hashed medical data. One or more differences can include changes to patient information, treatment plans, diagnosis, tests, facilities where tests were performed, and/or any other changes to data that can be included in a medical record. In this way, contextual data can also be searched via the search system and/or the database. In some implementations, third party entities that have been approved to access the database can set notifications when hashed medical records are updated, hashed medical data is newly-created, and/or certain contextual information satisfies a criteria established by the third-party entity. This can preserve network bandwidth for the search system, which might otherwise be consumed by users that frequently input similar queries.

The method 400 can proceed from the operation 408 to an operation 410, which can include determining whether a query was received at an interface of the search system. A query can be a natural language input to a search interface of a computer device that has been approved to access the database of the search system. For example, the search interface can be a text input field of an application that is executing at the computing device. Therefore, a user that is seeking to search via the search interface can provide a textual input to the text field in order to execute a search. Alternatively, or additionally, a search interface can include one or more interactive graphical elements for navigating to one or more portions of medical data in order to identify similar medical data that is stored or otherwise accessible via the database. For example, a medical professional may be accessing the search system in order to identify a patient that is similar to a particular patient of the medical professional. In order to effectuate a search for similar patients, the medical professional can identify a medical record of the particular patient to submit as a search query to the search system. When the query is determined to be provided to the interface of the search system, the method 400 can proceed from the operation 410 to an operation 412. Otherwise, the method 400 can proceed from the operation 410 to the operation 402.

The operation 412 can include identifying one or more portions of the hashed medical data that are similar to hashed query data. Identifying one or more portions of the hash medical data that are similar to the hashed query data can involve comparing features of instances of hashed medical data that are similar to features of the hashed query data. For example, one or more portions of hashed medical data can include multiple strings of natural language content of a finite length (e.g., N characters, where N is any number). The hashed query data can be considered similar to a string of hashed medical data when one or more characters are arranged in the hashed query data similar to how one or more characters are arranged in a string of the hashtag medical data. Specifically, a first hashing of medical data can be considered more similar to the hashed query data than a second hashing when the first hashing includes a string of characters that are positioned in the hashed query data at the same position that the string of characters is positioned in the hash query data—but the second hashing of medical data does not include the string of characters at the same position. Alternatively, or additionally, a first hashing of medical data can be considered more similar to be hashed query data than a second hashing of medical data when the first hashing includes a first string of characters that are also included in the hash query data, but the second hashing includes a second string of characters that are also included in the hashed query data, but the first string of characters includes more characters than the second string of characters.

The method 400 can further include an operation 414 of causing the interface of the search system to render one or more search results based on identifying the one or more similar portions of hashed medical data. In some implementations, each result of the identified results can be ranked according to their similarity to the hashed query data. Therefore, a most similar result can be prioritized in the interface over a less similar result. In some implementations, the interface can limit the presented results to at least indicate a degree of similarity (e.g., Result 1=97% similarity, Result 2=87% similarity, etc.) to the hashed query data, in order to preserve patient data until the patient and/or third party entity has provided consent for the user to have full access to the result and/or non-hashed medical data that is of interest to the user.

For example, a user can select a result from a listing of results and, in response, the search system can generate a request for a third party entity that is the source of the result to provide additional information corresponding to the identified result. In other words, each result presented to the user would be presented because similarities between hash values have been identified. Therefore, in order for the user to access non hashed data corresponding to a selected result, the user would need permission to decrypt the result that was identified by the user. In some implementations, an application that provides access to the search system can decrypt the selected result. Alternatively, or additionally in response to the user selecting a result, the search system can request that a third party entity that generated the medical data corresponding to the result decrypt the result for the user. When the user has completed research or otherwise received results, the method 400 can proceed to the operation 402 for monitoring for updated and/or new medical data provided to the database.

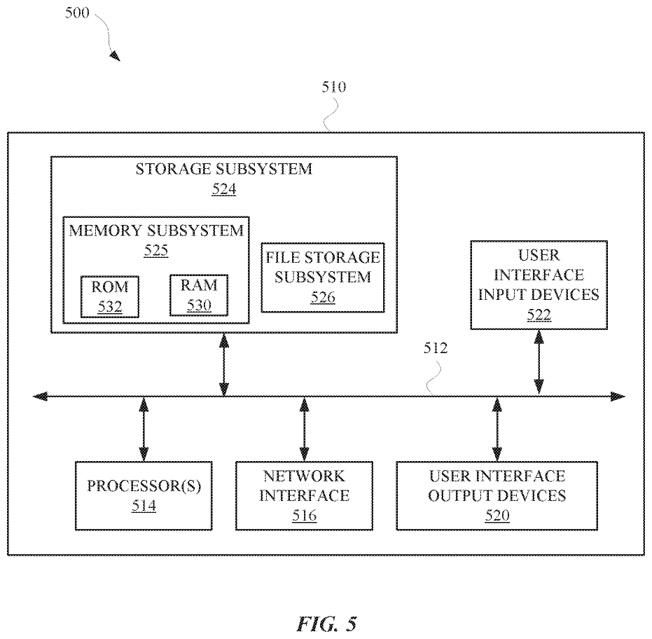

FIG. 5 is a block diagram of an example computer system 510. Computer system 510 typically includes at least one processor 514 which communicates with a number of peripheral devices via bus subsystem 512. These peripheral devices may include a storage subsystem 524, including, for example, a memory 525 and a file storage subsystem 526, user interface output devices 520, user interface input devices 522, and a network interface subsystem 516. The input and output devices allow user interaction with computer system 510. Network interface subsystem 516 provides an interface to outside networks and is coupled to corresponding interface devices in other computer systems.

User interface input devices 522 may include a keyboard, pointing devices such as a mouse, trackball, touchpad, or graphics tablet, a scanner, a touchscreen incorporated into the display, audio input devices such as voice recognition systems, microphones, and/or other types of input devices. In general, use of the term “input device” is intended to include all possible types of devices and ways to input information into computer system 510 or onto a communication network.

User interface output devices 520 may include a display subsystem, a printer, a fax machine, or non-visual displays such as audio output devices. The display subsystem may include a cathode ray tube (CRT), a flat-panel device such as a liquid crystal display (LCD), a projection device, or some other mechanism for creating a visible image. The display subsystem may also provide non-visual display such as via audio output devices. In general, use of the term “output device” is intended to include all possible types of devices and ways to output information from computer system 510 to the user or to another machine or computer system.

Storage subsystem 524 stores programming and data constructs that provide the functionality of some or all of the engines described herein. For example, the storage subsystem 524 may include the logic to perform selected aspects of method 400, and/or to implement one or more of search system database 110, first computing device 102, second computing device 104, computing device 212, search system database 202, third party database 228, search application 206, search system server 310, system 300, client device 320, and/or any other application, device, apparatus, and/or engine discussed herein.

These software engines are generally executed by processor 514 alone or in combination with other processors. Memory 525 used in the storage subsystem 524 can include a number of memories including a main random access memory (RAM) 530 for storage of instructions and data during program execution and a read only memory (ROM) 532 in which fixed instructions are stored. A file storage subsystem 526 can provide persistent storage for program and data files, and may include a hard disk drive, a floppy disk drive along with associated removable media, a CD-ROM drive, an optical drive, or removable media cartridges. The engines implementing the functionality of certain implementations may be stored by file storage subsystem 526 in the storage subsystem 524, or in other machines accessible by the processor(s) 514.

Bus subsystem 512 provides a mechanism for letting the various components and subsystems of computer system 510 communicate with each other as intended. Although bus subsystem 512 is shown schematically as a single bus, alternative implementations of the bus subsystem may use multiple busses.

Computer system 510 can be of varying types including a workstation, server, computing cluster, blade server, server farm, or any other data processing system or computing device. Due to the ever-changing nature of computers and networks, the description of computer system 510 depicted in FIG. 5 is intended only as a specific example for purposes of illustrating some implementations. Many other configurations of computer system 510 are possible having more or fewer components than the computer system depicted in FIG. 5.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be understood that certain expressions and reference signs used in the claims pursuant to Rule 6.2(b) of the Patent Cooperation Treaty (“PCT”) do not limit the scope.

In some implementations, a method implemented by one or more processors is set forth as including operations such as generating, at a server device and using one or more hashing functions, hashed medical data from medical records created at one or more computing devices. A first medical record of the medical records can be created at a first computing device (302) for a first patient and a second medical record of the medical records can be created at a second computing device (304) for a second patient. Furthermore, the first computing device can be connected to a local area network that is different from another local area network that to which the second computing device is connected. The method can further include, subsequent to generating the hashed medical data, receiving, via a client computing device, hashed search query data that is generated using the one or more hashing functions and query data that is input to a search interface. The one or more hashing functions can include a similarity preserving hashing function. The method can further include determining, in response to receiving the hashed search query data, whether the hashed search query data is similar to one or more portions of the hashed medical data, and causing, based on determining whether the hashed search query data is similar to one or more portions of hashed medical data, the client computing device to render one or more search results that are based on one or more particular hashed medical records generated from the medical records.

In some implementations, hashed query data is hashed at the client computing device using the one or more hashing functions, and the hashed query data is transmitted to the server device by the client computing device. In some implementations, the one or more particular hashed medical records are hashed according to the one or more hashing functions. In some implementations, the method can further include, subsequent to generating the hashed medical data, receiving, via a graphical user interface of the client computing device, a selection of a search result of the search results rendered at the client computing device, and causing, in response to receiving the selection of the search result, the client computing device to request a version of a particular hashed medical record of the one or more particular hashed medical records from a third party computing device.

In some implementations, a method implemented by one or more processors is set forth as including operations such as generating, at a server device and using one or more hashing functions, hashed medical data from medical records created at one or more computing devices. The method can further include determining, subsequent to generating the hashed medical data, that at least one medical record of the medical records has been modified subsequent to the medical records being created at the one or more computing devices. The method can also include generating, based on determining that the at least one medical record has been modified, additional hashed medical data. The hashed medical data can be stored with the additional hashed medical data, which is accessible to a user via a search interface of a client computing device. The method can further include, subsequent to generating the hashed medical data and the additional hashed medical data, receiving, via the client computing device, hashed search query data that is generated using the one or more hashing functions and query data that is input to a search interface of the client computing device by the user. The method can further include determining, in response to receiving the hashed search query data, whether the hashed search query data is similar to one or more portions of the hashed medical data. The method can further include causing, based on determining whether the hashed search query data is similar to one or more portions of hashed medical data, the client computing device to render one or more search results that are based on one or more particular hashed medical records included in the additional hashed medical data.

In some implementations, the one or more hashing functions include a similarity preserving hashing function. In some implementations, a first medical record of the medical records is created at a first computing device for a first patient and a second medical record of the medical records is created at a second computing device for a second patient. In some implementations, the first patient is different from the second patient, and the first computing device is connected to a local area network that is different from another local area network that the second computing device is connected to. In some implementations, the hashed query data is hashed at the client computing device using the one or more hashing functions, and the hashed query data is transmitted to the server device by the client computing device. In some implementations, the one or more particular hashed medical records are hashed according to the one or more hashing functions. In some implementations, the method can further include, subsequent to generating the hashed medical data and the additional hashed medical data, receiving, via a graphical user interface of the client computing device, a selection of a search result of the search results rendered at the client computing device, and causing, in response to receiving the selection of the search result, the client computing device to request a version of a particular hashed medical record of the one or more particular hashed medical records from a third party computing device.

In yet other implementations, a system is set forth as including one or more processors; and memory configured to store instructions that, when executed by the one or more processors, cause the one or more processors to perform operations. The operations can include generating, using one or more hashing functions, hashed medical data from medical records created at one or more computing devices. A first medical record of the medical records can be created at a first computing device for a first patient and a second medical record of the medical records can be created at a second computing device for a second patient. The first computing device can be connected to a local area network that is different from another local area network that to which the second computing device is connected. In some implementations, the operations can further include, subsequent to generating the hashed medical data: receiving, via a client computing device, hashed search query data that is generated using the one or more hashing functions and query data that is input to a search interface by a user. In some implementations, the operations can further include determining, in response to receiving the hashed search query data, whether the hashed search query data is similar to one or more portions of the hashed medical data. In some implementations, the operations can further include causing, based on determining whether the hashed search query data is similar to one or more portions of hashed medical data, the client computing device to render one or more search results that are based on one or more particular hashed medical records generated from the medical records.

In some implementations, the hashed query data is hashed at the client computing device using the one or more hashing functions. In some implementations, the one or more particular hashed medical records are hashed according to a similarity preserving hash function. In some implementations, the method can further include, subsequent to generating the hashed medical data: receiving, via a graphical user interface of the client computing device, a selection of a search result of the search results rendered at the client computing device, and causing, in response to receiving the selection of the search result, the client computing device to request a version of a particular hashed medical record of the one or more particular hashed medical records from a third party computing device.

What is claimed is:

1. A method implemented by one or more processors, the method comprising:
generating, at a server device and using one or more hashing functions, hashed medical data from medical records created at one or more computing devices;
wherein a first medical record of the medical records is created at a first computing device for a first patient and a second medical record of the medical records is created at a second computing device for a second patient, and
wherein the first computing device is connected to a local area network that is different from another local area network that to which the second computing device is connected;
subsequent to generating the hashed medical data:
receiving, via a client computing device, hashed search query data that is generated using the one or more hashing functions and query data that is input to a search interface,
wherein the one or more hashing functions include a similarity preserving hashing function,
determining, in response to receiving the hashed search query data, whether the hashed search query data is similar to one or more portions of the hashed medical data, and
causing, based on determining whether the hashed search query data is similar to one or more portions of hashed medical data, the client computing device to render one or more search results that are based on one or more particular hashed medical records generated from the medical records.

2. The method of claim 1, wherein the hashed query data is hashed at the client computing device using the one or more hashing functions, and the hashed query data is transmitted to the server device by the client computing device.

3. The method as in claim 1, wherein the one or more particular hashed medical records are hashed according to the one or more hashing functions.

4. The method as in claim 1, further comprising:
subsequent to generating the hashed medical data:
receiving, via a graphical user interface of the client computing device, a selection of a search result of the search results rendered at the client computing device, and
causing, in response to receiving the selection of the search result, the client computing device to request a version of a particular hashed medical record of the one or more particular hashed medical records from a third party computing device.

5. A method implemented by one or more processors, the method comprising:
generating, at a server device and using one or more hashing functions, hashed medical data from medical records created at one or more computing devices;
determining, subsequent to generating the hashed medical data, that at least one medical record of the medical records has been modified subsequent to the medical records being created at the one or more computing devices;
generating, based on determining that the at least one medical record has been modified, additional hashed medical data,
wherein the hashed medical data is stored with the additional hashed medical data, which is accessible to a user via a search interface of a client computing device; and
subsequent to generating the hashed medical data and the additional hashed medical data:
receiving, via the client computing device, hashed search query data that is generated using the one or more hashing functions and query data that is input to a search interface of the client computing device by the user,
determining, in response to receiving the hashed search query data, whether the hashed search query data is similar to one or more portions of the hashed medical data, and
causing, based on determining whether the hashed search query data is similar to one or more portions of hashed medical data, the client computing device to render one or more search results that are based on one or more particular hashed medical records included in the additional hashed medical data.

6. The method of claim 5, wherein the one or more hashing functions include a similarity preserving hashing function.

7. The method as in claim 5, wherein a first medical record of the medical records is created at a first computing device for a first patient and a second medical record of the medical records is created at a second computing device for a second patient.

8. The method of claim 7, wherein the first patient is different from the second patient, and wherein the first computing device is connected to a local area network that is different from another local area network that the second computing device is connected to.

9. The method as in claim 5, wherein the hashed query data is hashed at the client computing device using the one or more hashing functions, and the hashed query data is transmitted to the server device by the client computing device.

10. The method as in claim 5, wherein the one or more particular hashed medical records are hashed according to the one or more hashing functions.

11. The method as in claim 5, further comprising:
subsequent to generating the hashed medical data and the additional hashed medical data:
receiving, via a graphical user interface of the client computing device, a selection of a search result of the search results rendered at the client computing device, and
causing, in response to receiving the selection of the search result, the client computing device to request a version of a particular hashed medical record of the one or more particular hashed medical records from a third party computing device.

12. A system, comprising:
one or more processors; and
memory configured to store instructions that, when executed by the one or more processors, cause the one or more processors to perform operations that include:
generating, using one or more hashing functions, hashed medical data from medical records created at one or more computing devices;
wherein a first medical record of the medical records is created at a first computing device for a first patient and a second medical record of the medical records is created at a second computing device for a second patient, and
wherein the first computing device is connected to a local area network that is different from another local area network that to which the second computing device is connected;
subsequent to generating the hashed medical data:
receiving, via a client computing device, hashed search query data that is generated using the one or more hashing functions and query data that is input to a search interface by a user,
determining, in response to receiving the hashed search query data, whether the hashed search query data is similar to one or more portions of the hashed medical data, and
causing, based on determining whether the hashed search query data is similar to one or more portions of hashed medical data, the client computing device to render one or more search results that are based on one or more particular hashed medical records generated from the medical records.

13. The system of claim 12, wherein the hashed query data is hashed at the client computing device using the one or more hashing functions.

14. The system as in claim 12, wherein the one or more particular hashed medical records are hashed according to a similarity preserving hash function.

15. The system as in claim 12, further comprising:
subsequent to generating the hashed medical data:
receiving, via a graphical user interface of the client computing device, a selection of a search result of the search results rendered at the client computing device, and
causing, in response to receiving the selection of the search result, the client computing device to request a version of a particular hashed medical record of the one or more particular hashed medical records from a third party computing device.
